(19) United States
(12) Patent Application Publication
(10) Pub. No.: US 2024/0156337 A1
(43) Pub. Date: May 16, 2024

(54) IMAGE INPAINTING OF IN-VIVO IMAGES

(71) Applicant: Given Imaging LTD., Yoqneam (IL)

(72) Inventors: Eyal Dekel, Haifa (IL); Alexandra Gilinsky, Haifa (IL)

(21) Appl. No.: 18/496,253

(22) Filed: Oct. 27, 2023

Related U.S. Application Data
(60) Provisional application No. 63/425,331, filed on Nov. 15, 2022.

(51) Int. Cl. A61B 1/04; A61B 1/00; A61B 5/00

(52) U.S. Cl. CPC A61B 1/041; A61B 1/000096; A61B 1/00016; A61B 5/4255

(57) ABSTRACT

A system for image inpainting of in-vivo images includes at least one processor and at least one memory storing instructions. The instructions, when executed by the processor(s), cause the system to access an in-vivo image of a portion of a gastrointestinal tract where the in-vivo image includes image regions to be reconstructed, process the in-vivo image by a trained image inpainting deep learning model to provide a reconstructed in-vivo image, and provide the reconstructed in-vivo image to a device for viewing by a medical professional.

### CROSS-REFERENCE TO RELATED APPLICATIONS

This application claims the benefit of and priority to U.S. Provisional Patent Application No. 63/425,331, filed on Nov. 15, 2022, which is hereby incorporated by reference herein in its entirety.

### TECHNICAL FIELD

The present disclosure relates in-vivo images and, more particularly, to image inpainting of in-vivo images.

### BACKGROUND

Capsule endoscopy (CE) allows examining of a GIT endoscopically. There are capsule endoscopy systems and methods that are aimed at examining a specific portion of the GIT, such as the small bowel (SB) or the colon. CE is a non-invasive procedure which does not require the patient to be admitted to a hospital, and the patient can continue most daily activities while the capsule is in his body.

For a typical CE procedure, the patient is referred to a procedure by a physician. The patient then arrives at a medical facility (e.g., a clinic or a hospital), to perform the procedure. The capsule, which is about the size of a multi-vitamin, is swallowed by the patient under the supervision of a health professional (e.g., a nurse or a physician) at the medical facility and the patient is provided with a wearable device, e.g., a sensor belt having a recorder in a pouch and a strap to be placed around the patient's shoulder. The wearable device typically includes a storage device. The patient may be given guidance and/or instructions and then released to his daily activities.

The capsule captures images as it travels naturally through the GIT. Images and additional data (e.g., metadata) are then transmitted to the recorder that is worn by the patient. The capsule is typically disposable and passes naturally with a bowel movement. The procedure data (e.g., the captured images or a portion of them and additional metadata) is stored on the storage device of the wearable device.

The procedure data is uploaded from the wearable device to a computing system, which has an engine software stored thereon. The procedure data is then processed by the engine to generate a compiled study. Typically, the number of images in the procedure data to be processed is of the order of tens of thousands, and the generated study typically includes thousands of images.

A reader (which may be the procedure supervising physician, a dedicated physician, or the referring physician) may access the study via a reader application. The reader then reviews the study, evaluates the procedure, and provides input via the reader application. Since the reader needs to review thousands of images, the reading time of a study may usually take between half an hour to an hour on average and the reading task may be tiresome. A report is then generated by the reader application based on the compiled study and the reader's input. On average, it may take an hour to generate a report. The report may include, for example, images of interest, e.g., images which are identified as including pathologies, selected by the reader; evaluation or diagnosis of the patient's medical condition based on the procedure's data (i.e., the study) and/or recommendations for follow up and/or treatment provided by the reader. The report may be then forwarded to the referring physician. The referring physician may decide on a required follow up or treatment based on the report.

### SUMMARY

The present disclosure relates to image inpainting of in-vivo images. To the extent consistent, any or all of the aspects, embodiments, and examples detailed herein may be used in conjunction with any or all of the other aspects or embodiments detailed herein.

In accordance with aspects of the present disclosure, a system for image inpainting of in-vivo images includes at least one processor and at least one memory storing instructions. The instructions, when executed by the at least one processor, cause the system to access an in-vivo image of a portion of a gastrointestinal tract where the in-vivo image includes image regions to be reconstructed, process the in-vivo image by a trained image inpainting deep learning model to provide a reconstructed in-vivo image, and provide the reconstructed in-vivo image to a device for viewing by a medical professional.

In various embodiments of the system, the image regions to be reconstructed include regions where gastrointestinal content blocked a view of gastrointestinal tract tissue.

In various embodiments of the system, the reconstructed in-vivo image includes regions of reconstructed gastrointestinal tract tissue for the regions where the gastrointestinal content blocked the view of the gastrointestinal tract tissue.

In various embodiments of the system, the portion of the gastrointestinal tract includes a small bowel, and the regions of reconstructed gastrointestinal tract issue include small bowel mucosa.

In various embodiments of the system, the image regions to be reconstructed are indicated by a medical professional.

In various embodiments of the system, the trained image inpainting deep learning model is trained using training images, where the training images include images of a gastrointestinal tract that have randomly-located regions of random shapes removed.

In accordance with aspects of the present disclosure, a method for image inpainting of in-vivo images includes accessing an in-vivo image of a portion of a gastrointestinal tract where the in-vivo image includes image regions to be reconstructed, processing the in-vivo image by a trained image inpainting deep learning model to provide a reconstructed in-vivo image, and providing the reconstructed in-vivo image to a device for viewing by a medical professional.

In various embodiments of the method, the image regions to be reconstructed include regions where gastrointestinal content blocked a view of gastrointestinal tract tissue.

In various embodiments of the method, the reconstructed in-vivo image includes regions of reconstructed gastrointestinal tract tissue for the regions where the gastrointestinal content blocked the view of the gastrointestinal tract tissue.

In various embodiments of the method, the portion of the gastrointestinal tract includes a small bowel, and the regions of reconstructed gastrointestinal tract issue include small bowel mucosa.

In various embodiments of the method, the image regions to be reconstructed are indicated by a medical professional.

In various embodiments of the method, the trained image inpainting deep learning model is trained using training images, where the training images include images of a gastrointestinal tract that have randomly-located regions of random shapes removed.

In accordance with aspects of the present disclosure, a processor-readable medium stores instructions which, when executed by at least one processor of a system, cause the system to access an in-vivo image of a portion of a gastrointestinal tract where the in-vivo image includes image regions to be reconstructed, process the in-vivo image by a trained image inpainting deep learning model to provide a reconstructed in-vivo image, and provide the reconstructed in-vivo image to a device for viewing by a medical professional.

In various embodiments of the processor-readable medium, the image regions to be reconstructed include regions where gastrointestinal content blocked a view of gastrointestinal tract tissue.

In various embodiments of the processor-readable medium, the reconstructed in-vivo image includes regions of reconstructed gastrointestinal tract tissue for the regions where the gastrointestinal content blocked the view of the gastrointestinal tract tissue.

In various embodiments of the processor-readable medium, the portion of the gastrointestinal tract includes a small bowel, and the regions of reconstructed gastrointestinal tract issue include small bowel mucosa.

In various embodiments of the processor-readable medium, the image regions to be reconstructed are indicated by a medical professional.

In various embodiments of the processor-readable medium, the trained image inpainting deep learning model is trained using training images, where the training images include images of a gastrointestinal tract that have randomly-located regions of random shapes removed.

Further details and aspects of exemplary embodiments of the present disclosure are described in more detail below with reference to the appended figures.

### BRIEF DESCRIPTION OF THE DRAWINGS

A better understanding of the features and advantages of the disclosed technology will be obtained by reference to the following detailed description that sets forth illustrative embodiments, in which the principles of the technology are utilized, and the accompanying drawings of which:

FIG. 1 is a diagram of a gastrointestinal tract (GIT);

FIG. 2 is a block diagram of an example of a system for analyzing medical images captured in-vivo via a Capsule Endoscopy (CE) procedure, in accordance with aspects of the disclosure;

FIG. 3 is a block diagram of an example of a computing system, in accordance with aspects of the disclosure;

FIG. 4 is a block diagram of an example of a machine learning system, in accordance with aspects of the disclosure;

FIG. 5 is an example of a masked in-vivo image, in accordance with aspects of the disclosure;

FIG. 6 is an example of a reconstructed image, in accordance with aspects of the disclosure;

FIG. 7 is diagram of an example of operations of a convolutional neural network, in accordance with aspects of the disclosure;

FIG. 8 is a block diagram of an example of training a convolutional neural network, in accordance with aspects of the disclosure; and

FIG. 9 is a flow diagram of an example of operations of applying an image inpainting deep learning model, in accordance with aspects of the disclosure.

### DETAILED DESCRIPTION

The present disclosure relates to image inpainting of in-vivo images. An in-vivo imaging device, such has a capsule endoscope, captures images of a gastrointestinal tract (“GIT”). The images may be reviewed by a medical professional (e.g., physician, specialist, imaging technician, or otherwise) to evaluate the health or condition of the GIT or of portions of the GIT. When the GIT contains content (such as food particles, bubbles, fecal matter, etc.), the view of the GIT tissue may be obscured such that an in-vivo image may not capture GIT tissue obscured by content. In various circumstances, an in-vivo image may not clearly show GIT tissue for other reasons, such as light glare, among other reasons.

Aspects of the present disclosure relate to processing in-vivo images of a GIT to reconstruct regions of GIT tissue that, e.g., are obscured by content or are unclear due to light glare or other reasons. As used herein, the term “image inpainting” means the reconstruction of missing portions of an image. Aspects of the present disclosure perform such processing and reconstruction using an image inpainting deep learning model.

In the following detailed description, specific details are set forth in order to provide a thorough understanding of the disclosure. However, it will be understood by those skilled in the art that the disclosure may be practiced without these specific details. In other instances, well-known methods, procedures, and components have not been described in detail so as not to obscure the present disclosure. Some features or elements described with respect to one system may be combined with features or elements described with respect to other systems. For the sake of clarity, discussion of same or similar features or elements may not be repeated.

To the extent consistent, any or all of the aspects, embodiments, and examples detailed herein may be used in conjunction with any or all of the other aspects or embodiments detailed herein.

Although the disclosure is not limited in this regard, discussions utilizing terms such as, for example, “processing,” “computing,” “calculating,” “determining,” “establishing,” “analyzing,” “checking,” or the like, may refer to operation(s) and/or process(es) of a processor, a computing platform, a computing system, or other electronic computing device, that manipulates and/or transforms data represented as physical (e.g., electronic) quantities within computing registers and/or memories into other data similarly represented as physical quantities within the computing registers and/or memories or other non-transitory information storage medium that may store instructions to perform operations and/or processes.

Although the disclosure is not limited in this regard, the terms “plurality” and “a plurality” as used herein may include, for example, “multiple” or “two or more.” The terms “plurality” or “a plurality” may be used throughout the specification to describe two or more components, devices, elements, units, parameters, or the like.

As used herein, the term “exemplary” means “an example” and is not intended to mean preferred. Unless explicitly stated, the methods described herein are not constrained to a particular order or sequence. Additionally, some of the described methods or elements thereof can occur or be performed simultaneously, at the same point in time, or concurrently.

Depending on the context, the term “GIT” may mean a portion of the gastrointestinal tract and/or the entirety of a gastrointestinal tract. Thus, disclosures relating to a GIT may apply to a portion of the GIT and/or the entirety of a GIT.

The terms “image” and “frame” may each refer to or include the other and may be used interchangeably in the present disclosure to refer to a single capture by an imaging device. For convenience, the term “image” may be used more frequently in the present disclosure, but it will be understood that references to an image shall apply to a frame as well. As used herein, a “set” of images means and includes any collection of images, including images that may be ordered, unordered, consecutive, and/or non-consecutive.

The present disclosure may refer to image regions that are “designated” or “indicated” for reconstruction. Such descriptions do not mean and are not intended to mean that such image definitely requires reconstruction. Rather, such descriptions mean and are intended to mean that an image has such a designation or indication.

The term “machine learning” means and includes any technique which analyzes existing data to learn a model between inputs and outputs in the existing data. The term “machine learning model” means and includes any implementation of the learned model, in software and/or hardware, that can receive new input data and that can predict/infer output data by applying the learned model to the new input data. Machine learning may include supervised learning and unsupervised learning, among other things. Examples of machine learning models include, without limitation, deep learning neural networks and support vector machines, among other things.

The following description refers to images captured by a capsule endoscopy device. However, the following description may apply to other manners of obtaining images of a GIT or portion of a GIT.

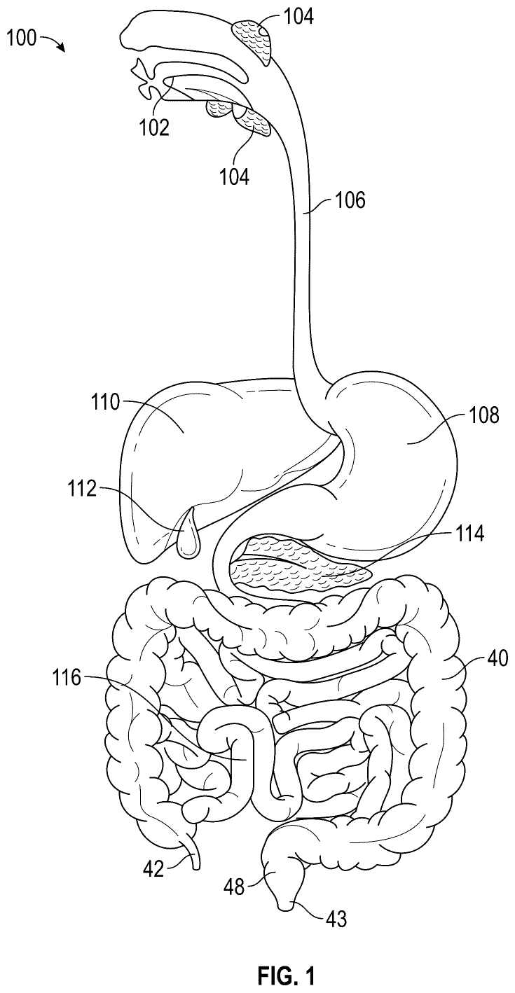

Referring to FIG. 1, an illustration of a gastrointestinal tract (GIT) 100 is shown. The GIT 100 is an organ system within humans and animals. The GIT 100 generally includes a mouth 102 for taking in sustenance, salivary glands 104 for producing saliva, an esophagus 106 through which food passes aided by contractions, a stomach 108 to secret enzymes and stomach acid to aid in digesting food, a liver 110, a gall bladder 112, a pancreas 114, a small intestine/small bowel 116 (“SB”) for the absorption of nutrients, and a colon 40 (e.g., large intestine) for storing water and waste material as feces prior to defecation. The colon 40 generally includes an appendix 42, a rectum 48, and an anus 43. Food taken in through the mouth is digested by the GIT to take in nutrients and the remaining waste is expelled as feces through the anus 43.

The type of procedure performed may determine which portion of the GIT 100 is the portion of interest. Examples of types of procedures performed include, without limitation, a procedure aimed to specifically exhibit or check the small bowel, a procedure aimed to specifically exhibit or check the colon, a procedure aimed to specifically exhibit or check the colon and the small bowel, or a procedure to exhibit or check the entire GIT: esophagus, stomach, SB, and colon, among other possibilities.

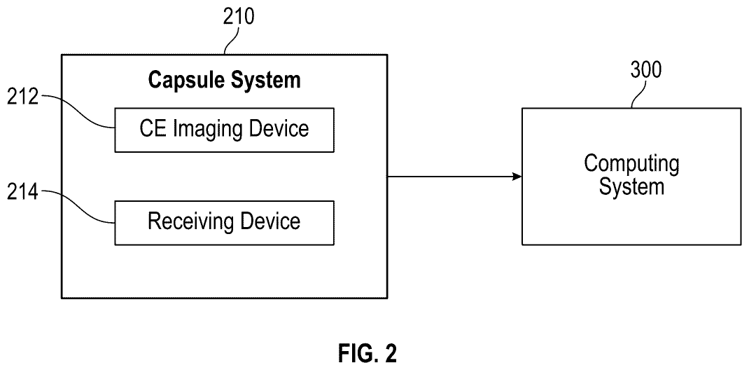

FIG. 2 shows a block diagram of a system for analyzing medical images captured in-vivo via a capsule endoscopy (“CE”) procedure. The system generally includes a capsule system 210 configured to capture images of the GIT and a computing system 300 (e.g., local system and/or cloud system) configured to process the captured images.

The capsule system 210 may include a swallowable CE imaging device 212 (e.g., a capsule) configured to capture images of the GIT as the CE imaging device 212 travels through the GIT. The images may be stored on the CE imaging device 212 and/or transmitted to a receiving device 214, typically via an antenna. In some capsule systems 210, the receiving device 214 may be located on the patient who swallowed the CE imaging device 212 and may, for example, take the form of a belt worn by the patient or a patch secured to the patient.

The capsule system 210 may be communicatively coupled with the computing system 300 and can communicate captured images to the computing system 300. The computing system 300 may process the received images using image processing technologies, machine learning technologies, and/or signal processing technologies, among other technologies. The computing system 300 may include local computing devices that are local to the patient and/or local to the patient's treatment facility, a cloud computing platform that is provided by cloud services, or a combination of local computing devices and a cloud computing platform.

In the case where the computing system 300 includes a cloud computing platform, the images captured by the capsule system 210 may be transmitted to the cloud computing platform. In various embodiments, the images can be transmitted by or via the receiving device 214 worn or carried by the patient. In various embodiments, the images can be transmitted via the patient's smartphone or via any other device which is connected to the Internet and which may be coupled with the CE imaging device 212 or the receiving device 214.

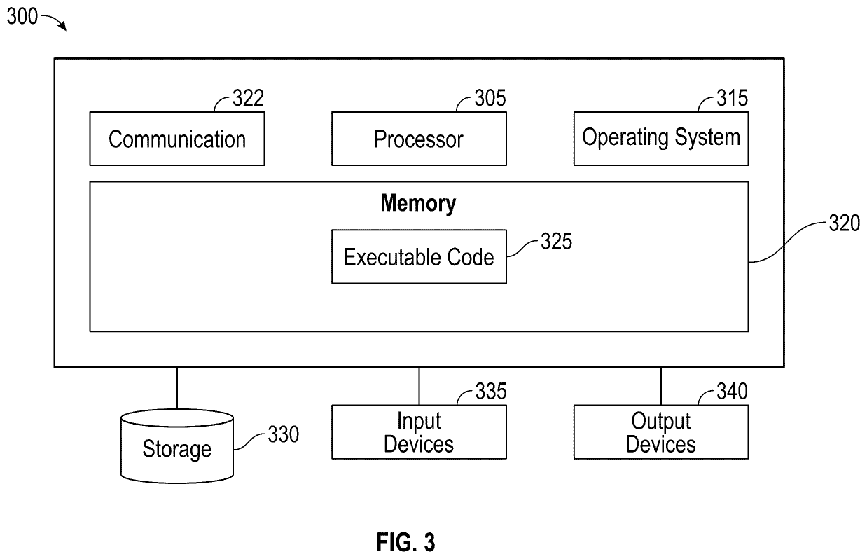

FIG. 3 shows a block diagram of example components of the computing system 300 of FIG. 2. The computing system 300 includes a processor 305, an operating system 315, a memory 320, a communication device 322, a storage 330, input devices 335, and output devices 340. The communication device 322 of the computing system 300 may allow communications with other systems or devices via a wired network (e.g., Ethernet) and/or a wireless network (e.g., Wi-Fi, cellular network, etc.).

The processor 305 may be or may include one or more central processing units (CPU), graphics processing unit (GPU), controllers, microcontrollers, microprocessors, and/or other computational devices. The operating system 315 may be or may include any code segment designed and/or configured to perform tasks involving coordination, scheduling, arbitration, supervising, controlling or otherwise managing operation of computing system 300, for example, scheduling execution of programs. Memory 320 may be or may include, for example, a Random Access Memory (RAM), a read-only memory (ROM), a Dynamic RAM (DRAM), a Synchronous DRAM (SD-RAM), a double data rate (DDR) memory chip, a Flash memory, a volatile memory, a non-volatile memory, a cache memory, a buffer, a short term memory, a long term memory, and/or other memory devices. The memory 320 stores executable code 325 that implements the data and operations of the present disclosure, which will be described later herein. Executable code 325 may be any executable code, e.g., an application, a program, a process, task, or script. Executable code 325 may be executed by the processor 305 possibly under control of operating system 315.

Storage 330 may be or may include, for example, a hard disk drive, a solid-state drive (SSD), a digital versatile disc (DVD), a universal serial bus (USB) device, and/or other removable and/or fixed device for storing electronic data. Instructions/code and data (e.g., images) may be stored in the storage 330 and may be loaded from the storage 330 into the memory 320, where it may be processed by processor 305.

Input devices 335 may include, for example, a mouse, a keyboard, a touch screen, and/or any other device that can receive an input. Output devices 340 may include one or more monitors, screens, displays, speakers, and/or any other device that can provide an output.

Other aspects of the computing system 300 and the capsule system (210, FIG. 2) are described in International Publication No. WO2020236683A1, entitled “Systems and Methods For Capsule Endoscopy Procedure,” which is hereby incorporated by reference in its entirety. Generally, the technology of the present disclosure may be utilized by capsule endoscopy systems or methods and may be presented in a user interface, such as the example user interfaces described in International Publication No. WO2020079696, entitled “Systems and Methods for Generating and Displaying a Study of a Stream of In-Vivo Images,” which is hereby incorporated by reference herein in its entirety.

The following description refers to images that may be part of a stream of images of the GIT and may be picked out or selected from the stream of GIT images. Small bowel images may be used merely as an example of the aspects and embodiments described below. The embodiments and aspects described herein also apply to other portions of a GIT, and it is intended that any description related to small bowel images shall be applicable to images of other portions of a GIT.

As mentioned above, aspects of the present disclosure relate to processing in-vivo images of a GIT to reconstruct regions of GIT tissue that, e.g., are obscured by content or are unclear due to light glare or other reasons. Aspects of the present disclosure perform such processing and reconstruction using an image inpainting deep learning model, which is described below.

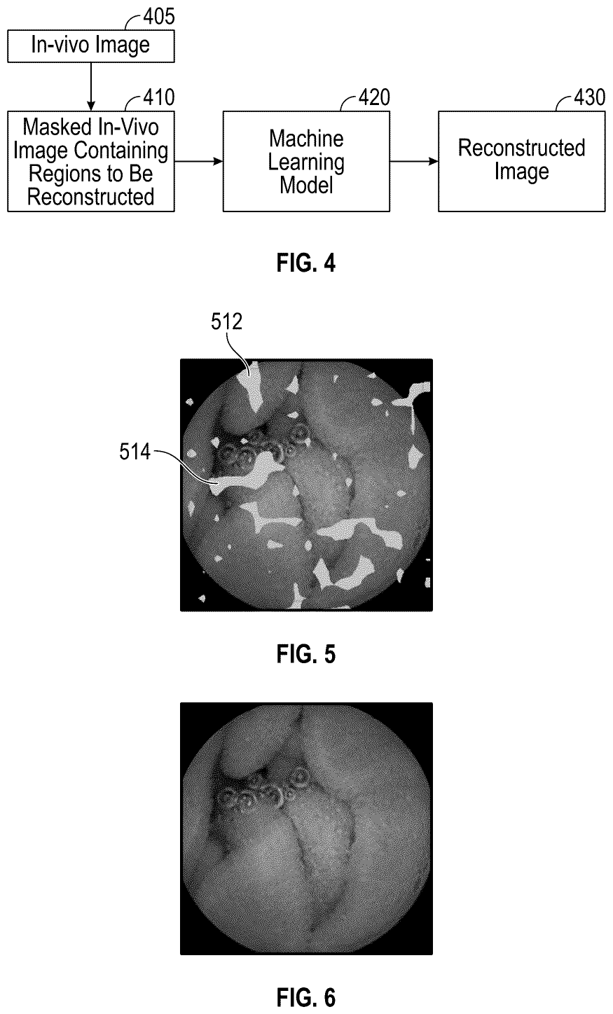

FIG. 4 is a block diagram of an example of a system in which a machine learning model 420 receives an in-vivo image containing regions to be reconstructed 410 and provides a reconstructed image 430. FIG. 4 illustrates an in-vivo image 405, which may be an image captured by a capsule endoscopy imaging device, such as the CE imaging device 212 of FIG. 2, or by another in-vivo imaging device. As mentioned above, the in-vivo image 405 may contain regions where GIT tissue is obscured by content and/or where GIT is unclear for another reason, e.g., light glare. The in-vivo image containing regions to be reconstructed 410 is a modified version of the in-vivo image 405 in which the regions of obscured or unclear GIT tissue are shown as missing and to be reconstructed. Such an image 410 may be referred to herein as a “masked in-vivo image.” The machine learning model 420 is trained to process the masked in-vivo image 410 to fill in the missing regions and to provide the reconstructed image 430.

The machine learning model 420 is implemented on a computing system, such as the computing system 300 of FIGS. 2 and 3, which may be a local system, a cloud system, or a combination thereof, as described above. The in-vivo image 405 and the masked in-vivo image 410 may be stored in such a computing system (e.g., in storage 330, FIG. 3) and/or may be communicatively sent to the computing system for processing by another device, such as by a medical professional workstation. The reconstructed image 430 may be stored in the computing system and/or may be communicated back to the device which sent the original images, e.g., the medical professional workstation.

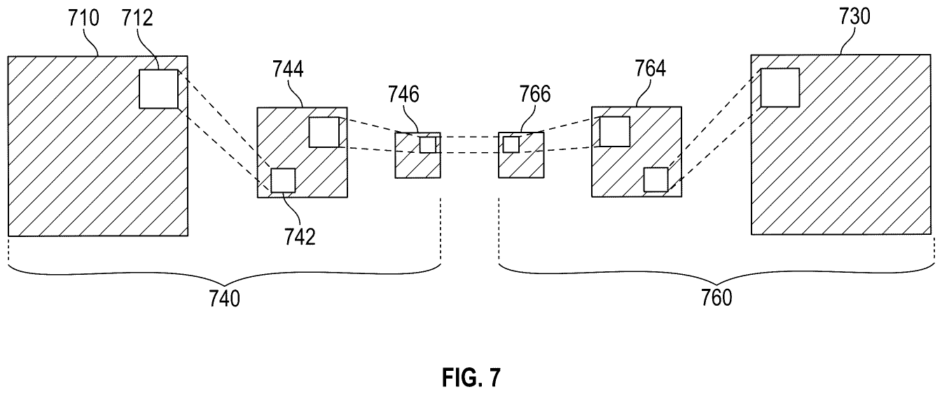

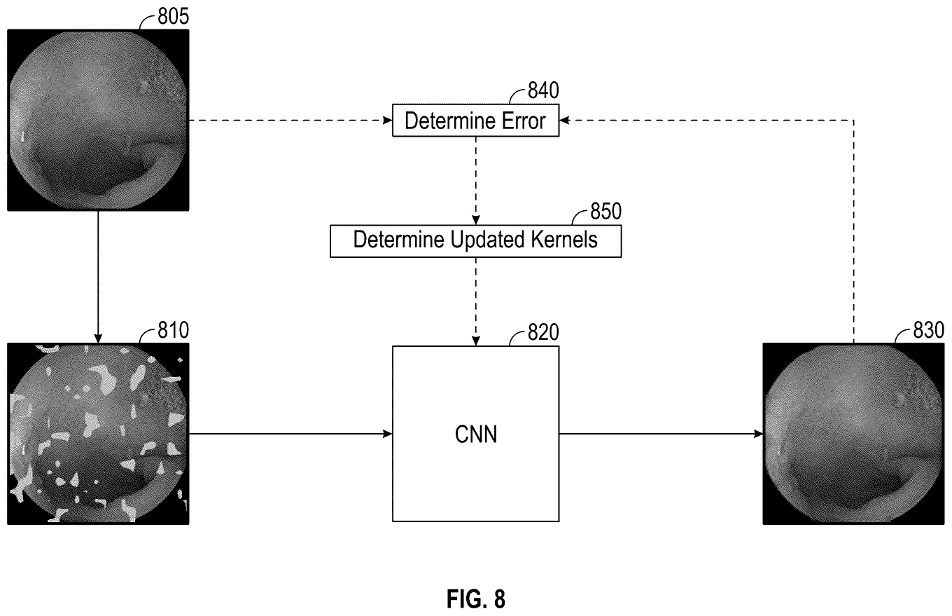

In the description below, examples of a masked in-vivo image and a reconstructed image are shown in FIGS. 5 and 6, respectively. The machine learning model 420 will be described in more detail in connection with FIG. 7. Training of the machine learning model will be described in connection with FIG. 8.

FIG. 5 shows an example of the masked in-vivo image 410 of FIG. 4. In FIG. 5, the regions 512, 514 are two of the regions in the image that are shown as missing and to be reconstructed. The regions 512, 514 are shown as missing using a designated color. In FIG. 5, a grey color is used in the regions 512, 514, but any other designated color may be used to show image regions as missing and to be reconstructed. The image of FIG. 5 is a masked small bowel image. This is merely an example, and in-vivo images of other portions of a GIT are within the scope of the present disclosure.

FIG. 6 shows an example of a result of the machine learning model (420, FIG. 4) reconstructing the missing regions of FIG. 5. In the reconstructed image of FIG. 6, the regions shown as missing in FIG. 5 have been filled in by a machine learning model (420, FIG. 4). The machine learning model will now be described below. The image of FIG. 6 is a reconstructed small bowel image in which missing regions of small bowel mucosa are reconstructed. This is merely an example, and in-vivo images of other portions of a GIT are within the scope of the present disclosure.

In accordance with aspects of the present disclosure, the machine learning model includes a convolution neural network (“CNN”). CNNs are a category of machine learning models that are suited for image processing. As persons skilled in the art will understand, CNNs process an image by performing computations on pixel values using deep learning techniques. The convolutional aspect of a CNN relates to applying matrix processing operations (called “kernels” or “filters”) to localized portions of an image. The kernels/filters are computationally adjusted during training of the CNN. A CNN typically includes convolution layers and pooling layers that reduce dimensionality without losing meaningful information.

FIG. 7 is a block diagram of a simplified example of convolutional neural network operations. FIG. 7 includes an input image 710 and an output image 730. The input image 710 may be the masked in-vivo image 410 of FIG. 4, and the output image 730 may be the reconstructed image 430 of FIG. 4. The operations of FIG. 7 may be implemented in a computing system, such as the computing system 300 of FIG. 2 and FIG. 3.

In the illustration, the CNN operations include kernel operations that are applied to an input image portion 712 of the input image 710, and the results of the kernel operations on the input image portion 712 are pooled into a smaller data set 742. When the kernel operations and pooling operations are applied to the entire input image 710, the resulting pooled data is shown in FIG. 7 as CNN layer 744. Further kernel and pooling operations can be applied to layer 744 to produce CNN layer 746. Altogether, the operations for processing the input image 710 to produce CNN layer 746 may be referred to herein as “encoding” operations 740. The illustrated and described encoding operations 740 are merely examples, and persons skilled in the art will recognize other operations that may be used in the encoding operations 740.

The encoding operations 740 may be reversed to produce the output image 730, and such operations may be referred to herein as “decoding” operations 760. The decoding operations 760 include performing deconvolution operations on the CNN layer 746 to produce CNN layer 766 and performing unpooling operations on CNN layer 766 to produce CNN layer 764. Further deconvolution and unpooling operations may be performed on CNN layer 764 to produce the output image 730. The illustrated and described decoding operations 760 are merely examples, and persons skilled in the art will recognize other operations that may be used in the decoding operations 760.

In accordance with aspects of the present disclosure, a CNN may be trained to perform image inpainting. As mentioned above, the term “image inpainting” means the reconstruction of missing portions of an image. FIG. 8 is a block diagram of an example training process. FIG. 8 includes a reference in-vivo image 805 in which GIT tissue is visible. The reference in-vivo image 805 may be a clean GIT image without content and without undesirable characteristics such as light glare, for example. The training process involves removing portions of the reference in-vivo image 805 to form a masked in-vivo image 810 and processing the masked in-vivo image 810 using the CNN 820 to produce a reconstructed image 830.

The training process involves determining an error 840 between the reconstructed image 830 and the reference in-vivo image 805 and, based on the error, using deep learning techniques to determine updated kernels 850 for the CNN 820. The updated kernels are then used in the CNN 820 for another iteration of the training process, and the process is repeated until training criteria are satisfied. Persons skilled in the art will understand how to implement deep learning techniques.

A plurality of reference in-vivo images and masked in-vivo images are used over the course of the training. In various embodiments, several thousand to hundreds of thousands of images may be used in the training process. At the outset of training, the error between the reconstructed image 830 and the reference in-vivo image 805 may be relatively large, such that the CNN 820 performs poorly at filling in the missing portions of the masked in-vivo image 810. As training progresses and the CNN is exposed to more images, the error level will decrease and the ability of the CNN 820 to appropriately fill in missing portions of the masked in-vivo image will improve.

In accordance with aspects of the present disclosure, the masked in-vivo images used in the training process (e.g., 810) are created from the reference in-vivo images (e.g., 805) by removing, from the reference in-vivo images, randomly-located portions of random shapes. It has been determined that a CNN's image inpainting ability is improved by training using masked in-vivo images containing randomly-located missing portions of random shapes.

FIG. 7 and FIG. 8 are merely examples. It is contemplated that image inpainting deep learning models, different from those shown and described herein, are within the scope of the present disclosure. For example, an image inpainting deep learning model may include aspects of the model described in Yan et al., “Shift-Net: Image Inpainting via Deep Feature Rearrangement,” European Conference on Computer Vision (2018), which is hereby incorporated by reference herein in its entirety. Persons skilled in the art will understand how to implement and train such models, including implementing configurations such as number of epochs, batch size, and loss functions (e.g., mean absolute error, mean squared error, etc.), among other parameters.

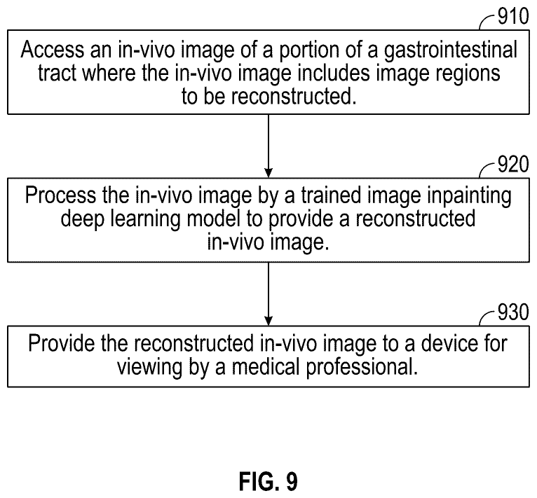

FIG. 9 is a flow diagram of an example of an operation for applying a trained image inpainting deep learning model. The operations of FIG. 9 may be implemented in a computing system, such as the computing system 300 of FIG. 2 and FIG. 3.

At block 910, the operation involves accessing an in-vivo image of a portion of a gastrointestinal tract where the in-vivo image includes image regions to be reconstructed. The image accessed at block 910 can be, for example, the masked in-vivo image 410 of FIG. 4. In various embodiments, the regions to be reconstructed in the masked in-vivo image may be indicated by a medical professional. For example, a medical professional may access an in-vivo image, such as in-vivo image 405 of FIG. 4, via a device and manually indicate portions of the in-vivo image to be removed and to be reconstructed. Such portions may include, for example, content that has blocked GIT tissue or characteristics such as light glare, among other things. The masked in-vivo image may be communicated to or upload to a computing system (e.g., 300, FIGS. 2 and 3) for processing by a trained image inpainting deep learning model.

At block 920, the operation involves processing the in-vivo image by a trained image inpainting deep learning model to provide a reconstructed in-vivo image. The image inpainting deep learning model may be a convolutional neural network with encoding and decoding operations, as described in connection with FIG. 7. In various embodiments, the image inpainting deep learning model may be the model described in Yan et al., “Shift-Net: Image Inpainting via Deep Feature Rearrangement,” European Conference on Computer Vision (2018), which was incorporated by reference above. The image inpainting deep learning model may be trained as described in connection with FIG. 8. The reconstructed image may be, for example, the reconstructed image 430 of FIG. 4.

At block 930, the operation involves providing the reconstructed in-vivo image to a device for viewing by a medical professional. The device may be the medical professional device which communicated or uploaded the masked in-vivo image to the computing system. In various embodiments, the reconstructed in-vivo image may be provided to the device upon request by the device. A medical professional may be able to provide a better evaluation of the health or condition of a patient's GIT by reviewing reconstructed in-vivo images free of content or other characteristics such as light glare.

The embodiments disclosed herein are examples of the disclosure and may be embodied in various forms. For instance, although certain embodiments herein are described as separate embodiments, each of the embodiments herein may be combined with one or more of the other embodiments herein. Specific structural and functional details disclosed herein are not to be interpreted as limiting, but as a basis for the claims and as a representative basis for teaching one skilled in the art to variously employ the present disclosure in virtually any appropriately detailed structure. Like reference numerals may refer to similar or identical elements throughout the description of the figures.

The phrases “in an embodiment,” “in embodiments,” “in various embodiments,” “in some embodiments,” or “in other embodiments” may each refer to one or more of the same or different embodiments in accordance with the present disclosure. A phrase in the form “A or B” means “(A), (B), or (A and B).” A phrase in the form “at least one of A, B, or C” means “(A); (B); (C); (A and B); (A and C); (B and C); or (A, B, and C).”

The systems, devices, and/or servers described herein may utilize one or more processors to receive various information and transform the received information to generate an output. The processors may include any type of computing device, computational circuit, or any type of controller or processing circuit capable of executing a series of instructions that are stored in a memory. The processor may include multiple processors and/or multicore central processing units (CPUs) and may include any type of device, such as a microprocessor, graphics processing unit (GPU), digital signal processor, microcontroller, programmable logic device (PLD), field programmable gate array (FPGA), or the like. The processor may also include a memory to store data and/or instructions that, when executed by the one or more processors, causes the one or more processors to perform one or more methods and/or algorithms.

Any of the herein described methods, programs, algorithms, or codes may be converted to, or expressed in, a programming language or computer program. The terms “programming language” and “computer program,” as used herein, each include any language used to specify instructions to a computer, and include (but is not limited to) the following languages and their derivatives: Assembler, Basic, Batch files, BCPL, C, C+, C++, Delphi, Fortran, Java, JavaScript, machine code, operating system command languages, Pascal, Perl, PL1, Python, scripting languages, Visual Basic, metalanguages which themselves specify programs, and all first, second, third, fourth, fifth, or further generation computer languages. Also included are database and other data schemas, and any other meta-languages. No distinction is made between languages which are interpreted, compiled, or use both compiled and interpreted approaches. No distinction is made between compiled and source versions of a program. Thus, reference to a program, where the programming language could exist in more than one state (such as source, compiled, object, or linked) is a reference to any and all such states. Reference to a program may encompass the actual instructions and/or the intent of those instructions.

It should be understood that the foregoing description is only illustrative of the present disclosure. Various alternatives and modifications can be devised by those skilled in the art without departing from the disclosure. Accordingly, the present disclosure is intended to embrace all such alternatives, modifications, and variances. The embodiments described with reference to the attached drawing figures are presented only to demonstrate certain examples of the disclosure. Other elements, steps, methods, and techniques that are insubstantially different from those described above and/or in the appended claims are also intended to be within the scope of the disclosure.

What is claimed:

1. A system for image inpainting of in-vivo images, the system comprising:
at least one processor; and
at least one memory storing instructions which, when executed by the at least one processor, cause the system to:
access an in-vivo image of a portion of a gastrointestinal tract, the in-vivo image comprising image regions to be reconstructed,
process the in-vivo image by a trained image inpainting deep learning model to provide a reconstructed in-vivo image, and
provide the reconstructed in-vivo image to a device for viewing by a medical professional.

2. The system of claim 1, wherein the image regions to be reconstructed comprise regions where gastrointestinal content blocked a view of gastrointestinal tract tissue.

3. The system of claim 2, wherein the reconstructed in-vivo image comprises regions of reconstructed gastrointestinal tract tissue for the regions where the gastrointestinal content blocked the view of the gastrointestinal tract tissue.

4. The system of claim 3, wherein the portion of the gastrointestinal tract comprises a small bowel, and
wherein the regions of reconstructed gastrointestinal tract issue comprise small bowel mucosa.

5. The system of claim 2, wherein the image regions to be reconstructed are indicated by a medical professional.

6. The system of claim 1, wherein the trained image inpainting deep learning model is trained using training images,
wherein the training images comprise images of a gastrointestinal tract that have randomly-located regions of random shapes removed.

7. A method for image inpainting of in-vivo images, the method comprising:
accessing an in-vivo image of a portion of a gastrointestinal tract, the in-vivo image comprising image regions to be reconstructed;
processing the in-vivo image by a trained image inpainting deep learning model to provide a reconstructed in-vivo image; and
providing the reconstructed in-vivo image to a device for viewing by a medical professional.

8. The method of claim 7, wherein the image regions to be reconstructed comprise regions where gastrointestinal content blocked a view of gastrointestinal tract tissue.

9. The method of claim 8, wherein the reconstructed in-vivo image comprises regions of reconstructed gastrointestinal tract tissue for the regions where the gastrointestinal content blocked the view of the gastrointestinal tract tissue.

10. The method of claim 9, wherein the portion of the gastrointestinal tract comprises a small bowel, and
wherein the regions of reconstructed gastrointestinal tract issue comprise small bowel mucosa.

11. The method of claim 8, wherein the image regions to be reconstructed are indicated by a medical professional.

12. The method of claim 7, wherein the trained image inpainting deep learning model is trained using training images,
wherein the training images comprise images of a gastrointestinal tract that have randomly-located regions of random shapes removed.

13. A processor-readable medium storing instructions which, when executed by at least one processor of a system, cause the system to:
access an in-vivo image of a portion of a gastrointestinal tract, the in-vivo image comprising image regions to be reconstructed;
process the in-vivo image by a trained image inpainting deep learning model to provide a reconstructed in-vivo image; and
provide the reconstructed in-vivo image to a device for viewing by a medical professional.

14. The processor-readable medium of claim 13, wherein the image regions to be reconstructed comprise regions where gastrointestinal content blocked a view of gastrointestinal tract tissue.

15. The processor-readable medium of claim 14, wherein the reconstructed in-vivo image comprises regions of reconstructed gastrointestinal tract tissue for the regions where the gastrointestinal content blocked the view of the gastrointestinal tract tissue.

16. The processor-readable medium of claim 15, wherein the portion of the gastrointestinal tract comprises a small bowel, and
wherein the regions of reconstructed gastrointestinal tract issue comprise small bowel mucosa.

17. The processor-readable medium of claim 14, wherein the image regions to be reconstructed are indicated by a medical professional.

18. The processor-readable medium of claim 13, wherein the trained image inpainting deep learning model is trained using training images,
wherein the training images comprise images of a gastrointestinal tract that have randomly-located regions of random shapes removed.
